(19) United States
(12) Patent Application Publication
(10) Pub. No.: US 2016/0157513 A1
(43) Pub. Date: Jun. 9, 2016

(54) FRUIT SNACKS FORTIFIED WITH POLYUNSATURATED FATTY ACIDS

(71) Applicant: WhiteWave Services, Inc., Denver, CO (US)

(72) Inventor: Angela Renee Kohut, Denver, CO (US)

(21) Appl. No.: 14/564,669

(22) Filed: Dec. 9, 2014

(51) Int. Cl. A23L 1/30; B29C 39/02; A61K 9/00; B29C 39/38; B29C 37/00; A61K 31/202; A23L 1/068; B29C 39/26

(52) U.S. Cl. CPC A23L 1/3008; A23L 1/068; B29C 39/02; B29C 39/26; B29C 39/38; B29C 37/0025; B29C 37/0032; A61K 31/202; A61K 9/0056; A23V 2002/00; B29K 2033/00

(57) ABSTRACT

According to some embodiments, a method comprises mixing a plurality of first ingredients into a slurry, cooking the slurry, and cooling the slurry to a temperature less than approximately 100° C. A polyunsaturated fatty acid (PUFA) is added to the cooled slurry, and the PUFA-fortified slurry is deposited into metal mold trays that have been coated with wax. The metal mold trays are cooled until the slurry sets into fruit snacks.

### TECHNICAL FIELD OF THE DISCLOSURE

Particular embodiments relate in general to fruit snacks and more particularly to fruit snacks fortified with polyunsaturated fatty acids.

### BACKGROUND

Polyunsaturated fatty acids (PUFAs) refer to a family of fatty acids that naturally occur in certain fish, leafy green vegetables, and vegetable oils. Polyunsaturated fatty acids can include a carbon chain comprising eighteen or more carbon atoms and two or more double bonds. Examples of polyunsaturated fatty acids include omega fatty acids, such as omega-3 fatty acids (e.g., docosahexaenoic acid (DHA), docosapentaenoic acid (n-3) (DPAn-3), stearidonic acid (SDA), linolenic acid (LNA), and alpha linoleic acid (ALA), and eicosapentaenoic acid (EPA)), and omega-6 fatty acids (e.g., arachidonic acid (ARA), docosapentaenoic acid (n-6) (DPAn-6), linoleic acid (LA), gamma linolenic acid (GLA), and dihomo gamma linolenic acid (n-6)). Research suggests that consuming certain polyunsaturated fatty acids can provide health benefits including possibly lowering the risk of heart disease. Accordingly, polyunsaturated fatty acids can be fortified into food products to impart health benefits. Fortifying certain food products with polyunsaturated fatty acids can tend to affect the flavor of the food product over time. For example, certain PUFAs may cause such food products to develop fishy or egg-like flavors.

### SUMMARY

According to some embodiments, a method comprises mixing a plurality of first ingredients into a slurry, cooking the slurry, and cooling the slurry to a temperature less than approximately 100° C. A polyunsaturated fatty acid (PUFA) is added to the cooled slurry, and the PUFA-fortified slurry is deposited into metal mold trays that have been coated with wax. The metal mold trays are cooled until the slurry sets into fruit snacks. The fruit snacks are then packaged.

According to one embodiment, a fruit snack contains approximately 32 mg DHA per 27 ounce serving of fruit snacks, a final moisture content of 18% to 30%, a dissolved sugar content of 75° Bx to 83° Bx, and an approximately 1:1 ratio of tapioca syrup to sugar.

Certain embodiments of the present disclosure can provide one or more technical advantages. For example, in some embodiments, a fruit snack can be fortified with a PUFA, such as DHA. The fruit snack may provide the nutrition of the PUFA in a format that has a child-friendly flavor and a gummy texture that allows the fruit snack to be formed into fun, custom shapes. A technical advantage of certain embodiments provides processes for protecting the PUFA to prevent oxidation. Known fruit snack manufacturing processes typical involve depositing a slurry in a starch mold and exposing the starch mold to air for 8-48 hours to allow the fruit snacks to properly set. After the fruit snacks set, the starch may be removed and the fruit snacks may be tumbled in wax to shine them up. This known process may be problematic for fruit snacks fortified with PUFAs because prolonged exposure to air may cause the PUFA to oxidize and develop off-flavors. Thus, certain embodiments provide a molding process that protects the PUFA.

For example, in certain embodiments a fruit snack slurry may have properties that allow it to set quickly such that the fruit snacks may be removed from the mold within one hour of the slurry being deposited in the mold. In certain embodiments, metal molds coated with wax may be used. When the fruit snacks are released from the metal molds, the wax may protect the PUFAs from oxidation. Because the wax is applied in the metal mold, the protective wax already coats the fruit snacks when they are released from the metal mold, which may allow the fruit snacks to be exposed to air for the remainder of the setting up/curing time without oxidizing. Once the fruit snacks have finished curing, they may be wrapped in packaging that helps protect the PUFA from oxidation.

By sufficiently protecting the PUFA, certain embodiments of the fruit snacks may be fortified with a relatively high amount of PUFA, such as at least approximately 32 mg per 27 ounce serving of fruit snacks, without the fruit snacks developing off-flavors over the shelf life of the product. For example, protecting the PUFA may allow for a shelf life of six months or more. In some embodiments, the PUFA used to fortify the fruit snacks can be an organic, algae-based PUFA compatible with organic, vegetarian, and vegan dietary habits.

Other technical advantages of the present disclosure will be readily apparent to one skilled in the art from the following figures, descriptions, and claims. Moreover, while specific advantages have been enumerated above, various embodiments can include all, some, or none of the enumerated advantages.

### BRIEF DESCRIPTION OF THE DRAWINGS

FIG. 1 illustrates an example of a system for producing fruit snacks with PUFAs;

FIG. 2 illustrates an example embodiment of the cooking system shown in FIG. 1;

FIG. 3 illustrates an example embodiment of the depositing line of FIG. 1; and

FIGS. 4A-4B illustrate a method for producing fruit snacks with PUFAs.

### DETAILED DESCRIPTION

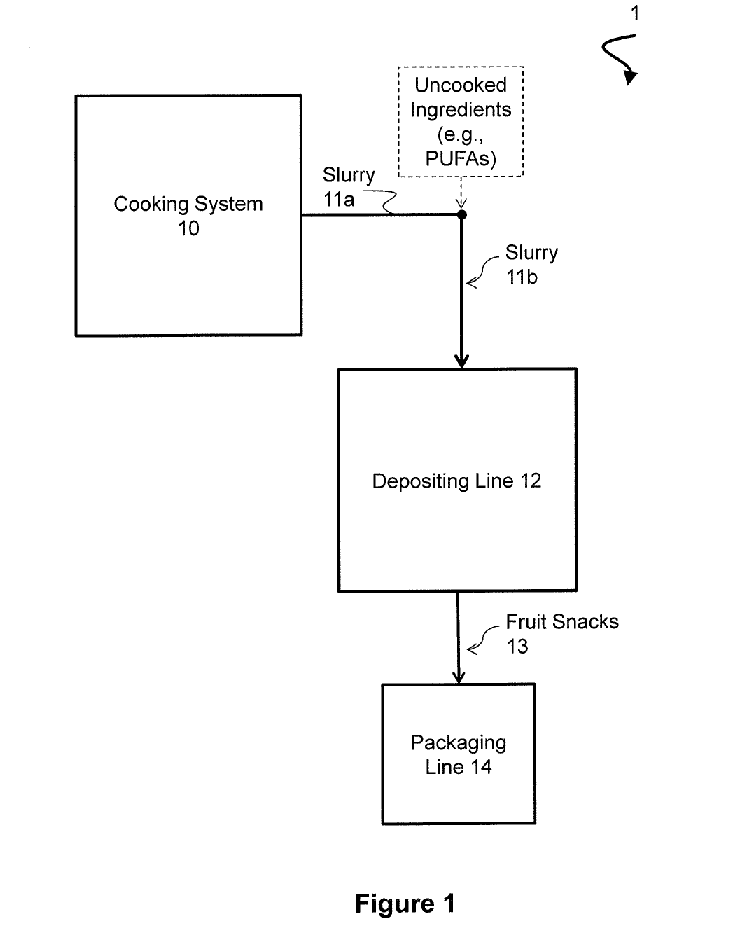

Embodiments of the present disclosure and its advantages are best understood by referring to FIGS. 1-4B of the drawings, like numerals being used for like and corresponding parts of the various drawings.

Polyunsaturated fatty acids (PUFAs) refer to a family of fatty acids that naturally occur in certain fish, green vegetables, algae, and breast milk. Research suggests that PUFAs may provide various health benefits, including fostering brain and cognitive growth in infants, maintaining ocular health, and treating diseases such as dementia and attention-deficit-hyperactivity-disorder. Thus, it may be desirable to fortify food products with PUFAs. Certain embodiments of the present disclosure provide methods for fortifying fruit snacks with PUFAs. Certain embodiments may minimize the exposure of the PUFAs to oxidizing agents, such as heat or air, in order to protect the stability of the PUFAs and prevent the fruit snacks from developing a fish-like flavor over the shelf life of the fruit snacks. By protecting the PUFAs, the fortified fruit snacks may have a shelf life of at least six months in certain embodiments.

FIG. 1 illustrates an embodiment of a system 1 for fortifying fruit snacks with PUFAs. For purposes of example, system 1 is described as fortifying the fruit snacks with docosahexaenoic acid (DHA). In alternative embodiments, any other suitable PUFA or combination of PUFAs may be used.

In some embodiments, system 1 can include a cooking system 10, depositing line 12, and packaging line 14. Cooking system 10 can introduce and mix certain ingredients in proportion, and cook the mixture at a proper temperature to yield slurry 11a. Examples of ingredients can include, but are not limited to, tapioca syrup, evaporated cane juice, fruit juices, water, pectin, and other solids and syrups. After exiting cooking system 10, slurry 11a may be mixed with one or more uncooked ingredients to form slurry 11b. Examples of the uncooked ingredients may include acids, natural colors, natural flavors, and DHA. Slurry 11b may be transported to depositing line 12 to be molded and set into fruit snacks 13. Fruit snacks 13 may then be packaged by packaging line 14. For example, packaging line 14 may wrap the fruit snacks 13 in a film and attach a label to fruit snacks 13 in preparation for eventual sale to consumers.

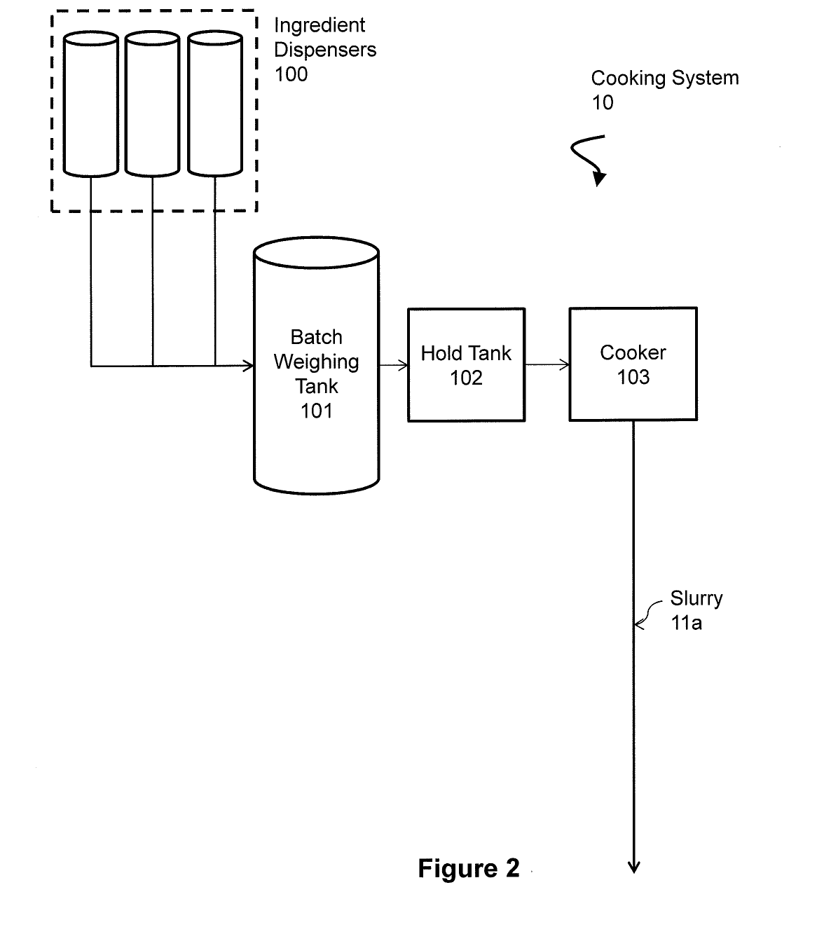

FIG. 2 illustrates an example of a cooking system 10, which comprises a plurality of ingredient dispensers 100, a batch weighing tank 101, a hold tank 102, and a cooker 103. The ingredient dispensers 100 act to supply certain ingredients and blends of ingredients for creating a slurry 11a. Examples of ingredient dispensers 100 include tanks that dispense liquid ingredients and hoppers that dispense dry ingredients. Pumps act to move metered amounts of liquid ingredients from the respective ingredient dispenser 100 to the batch weighing tank 101, which may be a thermograv in certain embodiments. The liquid and dry ingredients may be heated, cooled, blended, or subjected to any other suitable procedures to prepare them to be mixed. A dry ingredient can be either added to a liquid ingredient to create an ingredient blend, or a metered amount of the dry ingredient may be added directly from a dry ingredient dispenser 100 to the batch weighing tank 101. The batch weighing tank 101 blends the liquid and/or dry ingredients to achieve a consistently blended slurry 11a. Once the slurry 11a has exited the batch weighing tank 101, it passes through hold tank 102 to ensure that there is enough slurry (quantity) 11a to send to cooker 103 to keep a continuous flow. From hold tank 102, slurry 11a passes into cooker 103 to be cooked.

The selection of ingredients and the configuration of cooker 103 may be made to provide a balance among various factors, including flavor and texture, time before the slurry sets, final moisture content, cooking temperature, time to pass through the cooking system 10, and ratio of syrup to sugar. For example, increasing the sugar content and/or increasing the temperature in cooker 103 may tend to reduce the moisture content. In certain embodiments, the selection of ingredients and the configuration of cooker 103 may be made so as to create a slurry 11a that has sufficient moisture content to go through processing without setting inside the machinery, but not so much moisture as to require long setting times after being placed into molds. For example, conventional fruit snack setting times may be on the order of 8-48 hours, which may be problematic for DHA-fortified fruit snacks because prolonged exposure to air and/or heat may cause DHA to oxidize. Embodiments of the present disclosure may allow the fruit snacks to be released from the mold within less than approximately one hour after depositing the slurry in the mold. In addition, the fruit snacks may be coated in protective wax while in the mold in order to minimize the opportunity for the DHA to oxidize once the fruit snacks are released from the mold.

In certain embodiments, cooker 103 may cook slurry 11a at a temperature in the range of 100-120° C. In some embodiments, cooker 103 may apply vacuum to slurry 11a, for example, to further remove moisture. Any suitable amount of vacuum may be applied such as a value in the range of −0.5 to −1.0 bar, such as −0.70 bar to −0.8 bar, for example, −0.72 bar to −0.78 bar. In some embodiments, cooker 103 may be configured in order to achieve a slurry 11a having a Brix value in the range of 75° Bx to 83° Bx. The Brix value may represent dissolved sugar content and may be indicative of the fruit snack texture. In general, a slurry 11a having a lower Brix value may have a softer texture and may tend to take more time to set into a fruit snack, whereas a slurry 11a having a higher Brix value may have a firmer texture and may tend to take less time to set into a fruit snack. The Brix value, type of pectin, amount of pectin, type of buffer, amount of buffer, and/or other properties of slurry 11a can be adjusted to allow the slurry to set quickly enough to protect the DHA, but not so quickly that it sets inside the machinery (before being deposited into molds). In some embodiments, cooker 103 may be configured in order to achieve a final fruit snack moisture content of 18-30%.

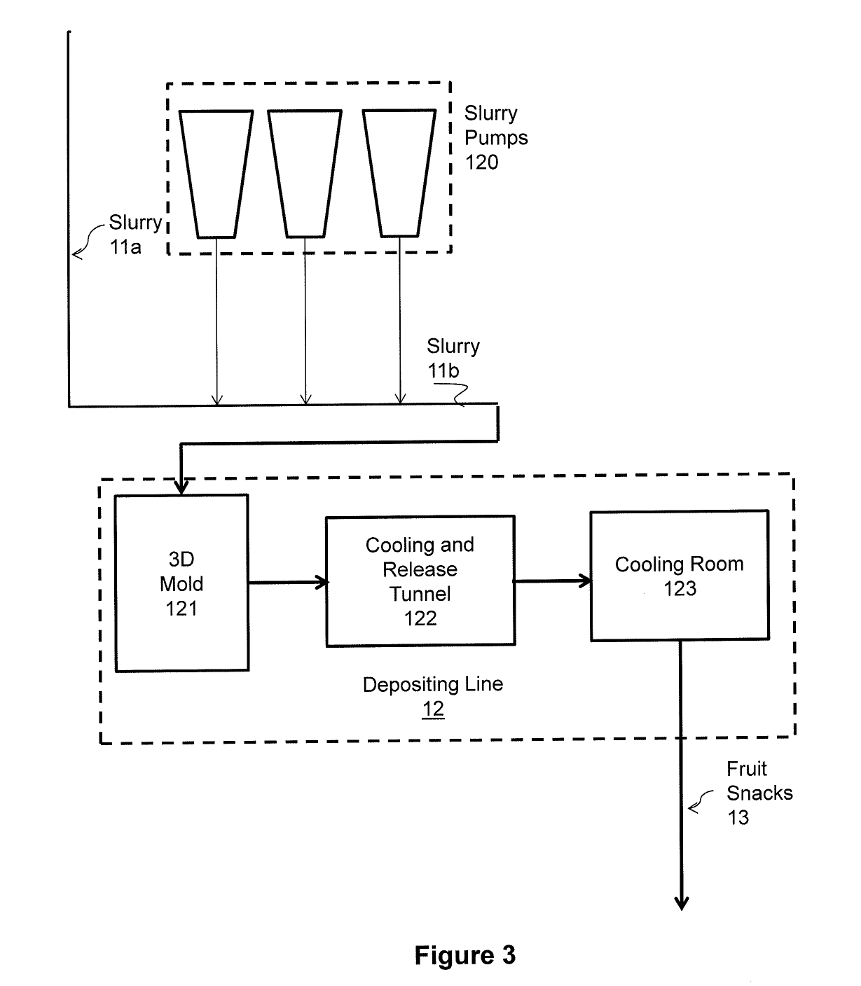

FIG. 3 illustrates an embodiment of a depositing line 12. The depositing line comprises a plurality of slurry pumps 120, a 3D mold 121, a cooling and release tunnel 122, and cooling room 123. Slurry pumps 120 contain ingredients which, for whatever reason, are unable or are not preferred to be added to the slurry prior to cooking. For example, DHA may be prone to developing off flavors when exposed to too much heat. Thus, slurry pumps may wait to introduce DHA until after slurry 11a has been cooked and cooled to a sufficiently cool temperature, such as approximately 100° C. (or less). Other examples of ingredients contained in slurry pumps 120 may include natural coloring, natural flavoring, and acids. In certain embodiments, multiple ingredients may be combined in a single slurry pump 120 when there are fewer slurry pumps 120 than ingredients.

DHA requires special handling and precautions when being added to slurry 11a. Due to DHA's heat sensitivity, certain embodiments involve storing the bulk DHA in freezers (not shown). In some embodiments, small portions of DHA are removed from the freezers as needed and are thawed at refrigeration or ambient temperatures until they are added to slurry 11a. In certain embodiments, DHA is added to the slurry 11a to a proportion of 32 mg of DHA per 27 ounce serving of fruit snacks.

Further, in embodiments requiring acids to be added to the slurry 11, especially certain no- or low-added sugar embodiments, a slurry pump 120 may contain acids or a blend of natural colors and acids. In embodiments where the acids are blended with natural colors, precautions can be taken to ensure that the natural colors are not undesirably affected by the addition of acids.

After all of the ingredients have been added, the slurry 11b is then deposited into a 3D mold 121, which comprises a metal tray formed to mold the slurry 11b into the final shape of the fruit snacks 13. Although known techniques for making fruit snacks commonly use starch molds, rather than metal molds, to shape the fruit snack, starch molds may be undesirable for making fruit snacks fortified with DHA. For example, DHA tends to leach into the starch causing the starch to oxidize and impart that flavor on the gummies. As a result, the starch must be thrown away more frequently, which may increase manufacturing costs. In addition, starch molds typically require fruit snacks to sit for one to two days to draw out extra moisture. However, it is undesirable for the DHA fortified slurry to sit out for that long, as the DHA may spoil without the protective wax coating that is applied during the molding process. Thus, in certain embodiments, metal trays may be used in order to speed the molding and setting process, which may reduce or eliminate DHA spoilage.

To reduce setting times, certain embodiments use pectin instead of gelatin because pectin sets up faster than gelatin. In certain embodiments using the 3D molding process with pectin, the fruit snacks 13 may set to the point where they release from the molds within 20 minutes, for example, the fruit snacks may be released between 7-10 minutes of being deposited in the molds and beginning the setting procedure. In some embodiments, the metal trays containing the pectin-containing slurry 11b may be sent through cooling and release tunnel 122 to set. In some embodiments, after spending about 10 minutes passing through cooling and release tunnel 122, the fruit snacks may be released from the metal trays. In some embodiments, wax is used in the molds as a release agent to prevent the fruit snacks 13 from sticking to the mold and to promote timely and full release of the chews from the mold. Using wax in the mold also helps preserve the DHA within the fruit snacks 13 by acting as a barrier between the chew (and its constituent DHA) and the outside air. The wax coating acts to reduce or prevent oxidation of the DHA, and thus reduce or prevent undesirable flavors from asserting themselves throughout the shelf life of the fruit snacks 13 as well as extending the shelf life of the chews.

Once the fruit snacks 13 fall out of the mold tray in the cooling and release tunnel 122, the fruit snacks 13 proceed to cooling room 123 or other suitable cooling area, where they cool for any suitable amount of time, such as approximately 4 to 72 hours, for example, 24 to 72 hours, such as 48 hours. In certain embodiments, the fruit snacks may optionally be tumbled in wax at some point during the cooling process. After cooling, the fruit snacks 13 may be sent to packaging line 14 and packaged in a film in order to maintain the freshness of the fruit snacks 13.

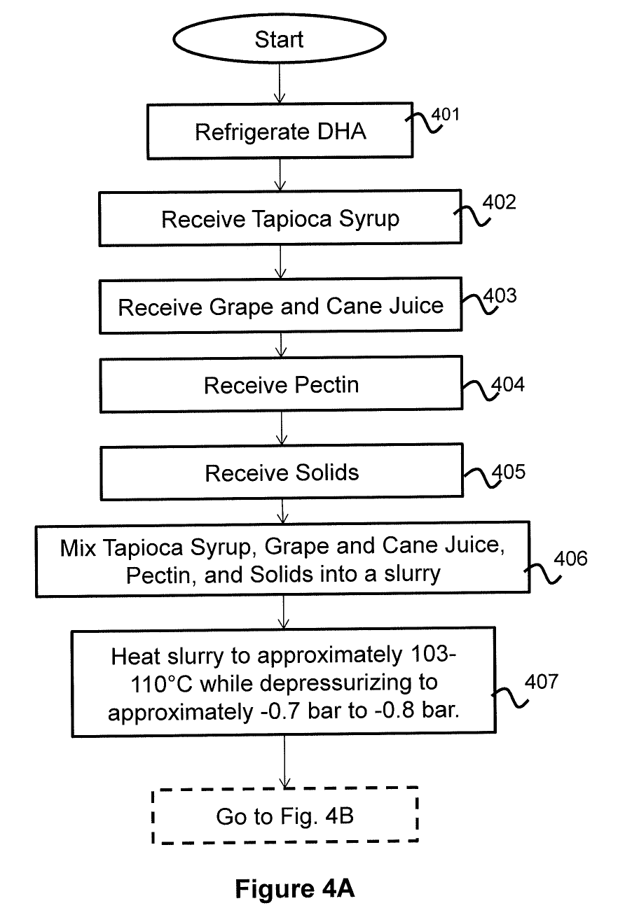

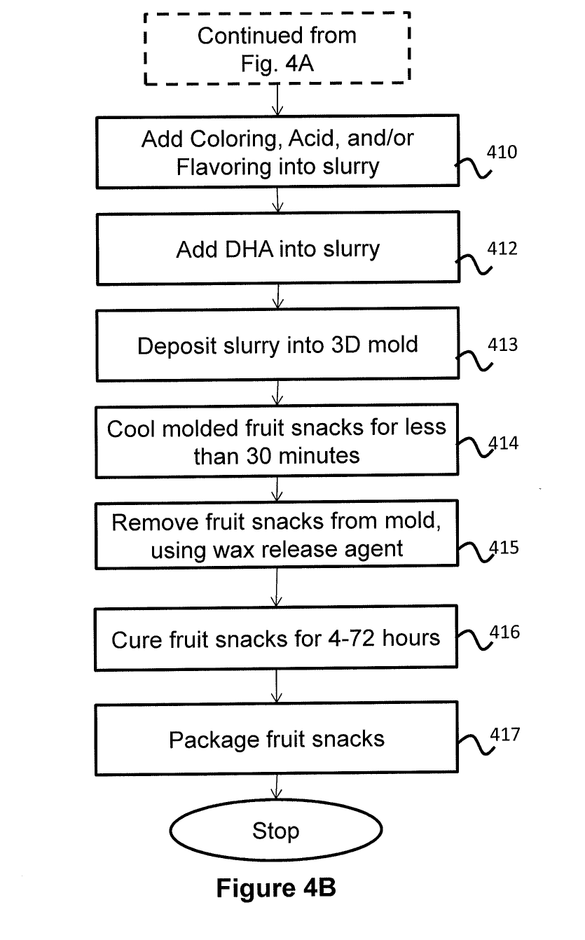

FIGS. 4A and 4B illustrate an example of a method to create PUFA-fortified fruit snacks. In general, the method mixes a plurality of first ingredients into a slurry, cooks the slurry, and cools the slurry. A PUFA (e.g., DHA) is added to the cooled slurry, and the PUFA-fortified slurry is deposited into metal mold trays and coated with wax. The metal mold trays are cooled until the slurry sets into fruit snacks. The fruit snacks are then cooled and packaged with nitrogen.

Turning to the example in FIG. 4A, the method begins at step 401 where a small portion of DHA is refrigerated in anticipation of being fortified into the fruit snacks. Prior to being placed on the production line, the DHA may be stored in freezers. Once the stored DHA is opened/thawed, it is preferable to use the DHA relatively soon. The DHA may be thawed at refrigeration or ambient temperatures for approximately 24 hours prior to being added to the fruit snack slurry. A small portion of DHA may be thawed at a time so that only the amount of DHA needed to continually add fresh/cold DHA throughout the day is thawed. This may prevent the DHA from getting or staying hot. This may also prevent waste as any unused DHA may have to be discarded (e.g., due to the sensitivity of DHA, it is generally not well-suited to refreezing after it has been opened and thawed). In some embodiments, the DHA is organic compliant. In those embodiments, the DHA may be derived from algae-based sources.

At steps 402-405, first ingredients are received and prepared to be mixed into a slurry. The first ingredients may include liquid and/or dry ingredients. In certain embodiments, the first ingredients comprise tapioca syrup, grape juice, evaporated cane juice, pectin, and other solids (such as dextrose). In steps 402-405, the first ingredients may be maintained at a specific temperature, blended together, or otherwise prepared to be mixed into a slurry.

The first ingredients are combined and mixed into a slurry in step 406. In certain embodiments, liquid ingredients may be metered into the batch weighing tank by pumps. Further, in some embodiments, dry ingredients may be automatically or manually metered into the batch weighing tank. In other embodiments, dry ingredients may be blended with liquid ingredients in a proportion to allow the blend to be metered into the batch weighing tank. The batch weighing tank mixes the first ingredients until they form a consistent, well-mixed slurry.

From the batch weighing tank, the method proceeds to step 407 where the slurry is cooked. For example, in certain embodiments the slurry is heated to a temperature in the range of 103° C. to 115° C. while being depressurized to a vacuum pressure in the range of −0.7 bar to −0.8 bar. The process of heating and depressurizing the slurry serves to reduce the moisture content to a level where at the end of the method, the final moisture content reaches 18-30%. In some embodiments, the slurry is cooked until it has a Brix value in the range of 75° Bx to 83° Bx and/or a moisture content between 18% and 30%. Thus, in certain embodiments, the moisture content when the slurry exits the cooking step may be similar to the final moisture content of the fruit snacks.

Continuing to FIG. 4B, steps 410-412 describe adding second ingredients into the slurry that are either unable, impractical, or for whatever reason otherwise not selected to add prior to cooking. Steps 410-412 may be performed after the slurry cools to approximately 100° C., and examples of second ingredients that may be added at steps 410-412 include natural coloring, natural flavoring, acids, and DHA. Each ingredient may be added from its own pump, or certain ingredients may be blended and added from a shared pump. In certain embodiments, a single pump may be used to pump one of the following combinations of ingredients: 1) coloring and acid, 2) coloring and flavoring, 3) flavoring and acid, or 4) coloring, acid, and flavoring. As shown in step 401, the DHA is refrigerated and kept cool until added into the slurry. DHA is highly heat sensitive, and keeping it cool during the production process acts to preserve the DHA and extend the fruit snacks' shelf life.

Once the second ingredients are added into the slurry, the method proceeds to step 413 where the slurry is deposited into a three-dimensional metal mold tray. Wax may be applied to the inside of the mold to protect the DHA and to aid release from the mold once the fruit snack has set. At step 414, the slurry in the metal mold tray is cooled to allow it to set into the fruit snacks. The metal mold tray requires less time to set the fruit snacks than the time that would be required to set the fruit snacks using a starch mold. For example, a starch mold typically requires 1 to 2 days to set the fruit snacks, by which time the DHA in the chews would spoil. By contrast, the fruit snacks may require less than approximately 30 minutes to set in a metal mold tray. Thus, using a metal mold tray limits the DHA fortified fruit snacks' exposure to potentially oxidizing outside air. In certain embodiments, the fruit snacks are cooled for approximately 10 minutes in a cooling tunnel and then released from the mold at step 415. In certain embodiments release of the fruit snacks from the mold is aided by a wax release agent.

After releasing from the mold, the fruit snacks may be cured at step 416 for any suitable amount of time, such as for 4 to 72 hours, for example, 24 to 72 hours, such as 48 hours. The fruit snacks may then proceed to the packaging line to be packaged at step 417. In some embodiments, the fruit snacks may be packaged in a nitrogen-flushed high barrier velum package. As an example, Curwood Curpolene® Grade L7171 protective packaging film from Bemis® Company may be used as the high barrier velum packaging.

Tables 1 to 4 below provide examples of formulas that may be used in the method described with respect to FIGS. 4A and 4B. In the examples, ingredients described as having 0% as a possible quantity may be optional depending on the embodiments. In the examples, ingredients listed as organic may be substituted with non-organic ingredients without departing from the scope of the disclosure.

Table 1 provides an example of first ingredients that may be mixed at step 406.

TABLE 1

| First Ingredients | Quantity of the Mixture |
|---|---|
| Juice | 8%-50% |
| Tapioca Syrup | 20%-35% |
| Evaporated Cane Juice | 0%-30% |
| Water | 15%-20% |
| Pectin | 1%-5% |
| Solids | 0%-15% |

The juice may include any suitable fruit-based and/or vegetable-based juice. Examples include, but are not limited to, white grape juice, grape juice, apple juice, pear juice, pineapple juice, berry juice, beet juice, carrot juice, or mixtures of juices. Examples of solids include corn syrup solids, tapioca solids, and starch.

Table 2 provides an example of second ingredients that may be added at step 410-412.

TABLE 2

| Second Ingredients | Quantity of the Cooled Slurry |
|---|---|
| PUFA (e.g., DHA) | 0.1%-3% |
| Acids | 0.1%-3% |
| Colorings and/or flavorings | 0.1%-5% |

Examples of acids include ascorbic acid, citric acid, lactic acid, and combinations thereof.

Examples of polyunsaturated fatty acids include omega fatty acids, such as omega-3 fatty acids (e.g., docosahexaenoic acid (DHA), docosapentaenoic acid (n-3) (DPAn-3), stearidonic acid (SDA), linolenic acid (LNA), and alpha linoleic acid (ALA), and eicosapentaenoic acid (EPA)), and omega-6 fatty acids (e.g., arachidonic acid (ARA), docosapentaenoic acid (n-6) (DPAn-6), linoleic acid (LA), gamma linolenic acid (GLA), and dihomo gamma linolenic acid (n-6)), and combinations thereof.

Table 3 provides an example of ingredients for fruit snacks:

TABLE 3

| Ingredients | Quantity of the Fruit snack |
|---|---|
| Organic White Grape Juice | 40%-50% |
| Organic Tapioca Syrup | 20%-25% |
| Organic Evaporated Cane Juice | 15%-25% |
| Pectin | 1.5%-3% |
| Buffered Lactic Acid | 0%-1% |
| DHA | 0.5%-1% |
| Salt(s) | 0%-1% |
| Citric Acid | 0%-1% |
| Color | 0%-2% |
| Flavor | 0%-2% |
| Water | 10%-20% |
| Ascorbic Acid | 0%-0.5% |
| Finishing Wax (e.g., beeswax) | <0.15% |

In certain embodiments, sodium citrate may be used as the salt. In other embodiments, one or more citrate salts, phosphate salts, or sodium chloride may be used as the salt.

Table 4 provides another example of ingredients for fruit snacks:

TABLE 4

| Ingredients | Quantity of the Fruit snack |
|---|---|
| Organic White Grape Juice | 5%-15% |
| Organic Tapioca Syrup | 25%-35% |
| Tapioca Solids | 5%-15% |
| Organic Evaporated Cane Juice | 25%-30% |
| Pectin | 1.5%-3% |
| DHA | 0.5%-1% |
| Citric Acid | 0.2%-0.8% |
| Color | 0%-2% |
| Flavor | 0%-2% |
| Water | 15%-25% |
| Ascorbic Acid | 0.1%-0.5% |
| Finishing Wax (e.g., beeswax) | <0.15% |

The systems, methods, and formulations described herein may provide a fruit snack fortified with PUFAs. In certain embodiments, wax may be applied relatively early in the manufacturing process to protect the PUFA from oxidation. The wax may be applied by coating the metal molds used to set the fruit snacks. The wax may have a dual purpose: 1) act as a release agent so gummies come out of the molds after cooling, and 2) provide a protective coating immediately upon release from the molds. The protective wax coating may allow the fruit snacks to be exposed to the air to finish setting up/curing for up to 72 hours without oxidizing. The fruit snacks may then be placed in packaging that helps protect the PUFA. In some embodiments, the packaging may be flushed with nitrogen, which may help achieve a desired shelf life of at least six months.

The use of metal molds coated with wax may provide a technical advantage compared to the use of starch molds. In starch molds, the starch is used to pull moisture from the formula and actually set up. The starch molds are porous, so the fruit snacks are exposed to air the entire time they are setting up/curing (e.g., 8-48 hours). After the starch is removed, the gummies can then be waxed to shine them up. Thus, while a starch mold process includes a significant amount of time where the fruit snacks are exposed to the air (which is problematic for PUFAs), the metal mold process described herein limits air exposure by providing the protection of the metal mold itself and then the wax coating early in the process.

Modifications, additions, or omissions may be made to the systems and apparatuses disclosed herein without departing from the scope of the disclosure. The components of the systems and apparatuses may be integrated or separated. Moreover, the operations of the systems and apparatuses may be performed by more, fewer, or other components. Modifications, additions, or omissions also may be made to the methods disclosed herein without departing from the scope of the disclosure. The methods may include more, fewer, or other steps. Additionally, steps may be performed in any suitable order.

Although the present disclosure has been described with several embodiments, numerous changes, variations, alterations, transformations, and modifications can be suggested to one skilled in the art, and it is intended that the present disclosure encompass such changes, variations, alterations, transformations, and modifications as falling within the scope of the appended claims.

1. A method for creating fruit snacks, comprising:
mixing a plurality of first ingredients into a slurry, the first ingredients comprising tapioca syrup in an amount between approximately 20% and 35% of the slurry, white grape juice in an amount between approximately 8% and 50% of the slurry, evaporated cane juice in an amount less than approximately 30% of the slurry, water in an amount between approximately 15% and 20% of the slurry, and pectin in an amount between approximately 1% and 5% of the slurry;
cooking the slurry at a temperature in the range of 103° C. to 115° C. and a vacuum pressure in the range of −0.7 bar to −0.8 bar until the slurry has a Brix value is in the range of 75° Bx to 83° Bx and a moisture content between 18% and 30%;
cooling the slurry to a temperature of approximately 100° C.;
adding a plurality of second ingredients to the cooled slurry, the plurality of second ingredients comprising refrigerated docosahexaenoic acid (DHA) in an amount between approximately 0.1% and 3% of the slurry, one or more acids in an amount between approximately 0.1% and 3% of the slurry, and one or more colorings or flavorings;
depositing the DHA-fortified slurry into metal mold trays that have been coated with wax;
cooling the metal mold trays until the slurry sets into fruit snacks;
releasing the fruit snacks from the metal mold trays within 30 minutes of depositing the PUFA-fortified slurry into the metal mold trays;
cooling the fruit snacks for approximately 24 to 72 hours after releasing the fruit snacks from the metal mold trays and prior to packaging the fruit snacks; and
packaging the fruit snacks in a nitrogen-flushed high barrier velum package.

2. A method for creating fruit snacks, comprising:
mixing a plurality of first ingredients into a slurry;
cooking the slurry;
cooling the slurry to a temperature less than approximately 100° C.;
adding a polyunsaturated fatty acid (PUFA) to the cooled slurry;
depositing the PUFA-fortified slurry into metal mold trays that have been coated with wax;
cooling the metal mold trays until the slurry sets into fruit snacks; and
packaging the fruit snacks.

3. The method of claim 2, wherein the first ingredients comprise tapioca syrup, white grape juice, evaporated cane juice, water, solids, and pectin.

4. The method of claim 2, wherein cooking the slurry comprises heating the slurry to a temperature in the range of 103° C. to 115° C. and applying a vacuum pressure in the range of −0.7 bar to −0.8 bar.

5. The method of claim 2, wherein the slurry is cooked until its Brix value is in the range of 75° Bx to 83° Bx.

6. The method of claim 2, wherein the slurry is cooked until its moisture content is in the range of 18% to 30%.

7. The method of claim 2, further comprising releasing the fruit snacks from the metal trays within 30 minutes of depositing the slurry in the metal trays.

8. The method of claim 2, wherein the fruit snacks are cooled for approximately 2 to 4 hours after release from the metal trays and prior to coating the fruit snacks with the wax.

9. The method of claim 2, wherein the fruit snacks are cooled for approximately 24 to 72 hours after coating the fruit snacks with wax and prior to packaging the fruit snacks in a nitrogen-flushed high barrier velum package.

10. The method of claim 2, further comprising: adding one or more second ingredients after cooling the slurry to the temperature less than 100° C. and prior to depositing the PUFA-fortified slurry into the metal mold trays, the second ingredients selected from the group consisting of lactic acid, citric acid, ascorbic acid, monosodium citrate, sodium citrate, colorings, and flavorings.

11. A fruit snack food product, comprising:
juice;
tapioca syrup;
pectin;
solids;
polyunsaturated fatty acid (PUFA); and
one or more acids selected from the group consisting of citric acid, ascorbic acid, and lactic acid;
wherein the polyunsaturated fatty acid is present in an amount of at least 20 mg per 27 ounce serving of the fruit snack food product; and
the fruit snack food product is coated in a finishing wax.

12. The fruit snack food product of claim 11, wherein:
the juice comprises white grape juice present in an amount between approximately 10%-50% of the fruit snack food product;
the tapioca syrup is present in an amount between approximately 20%-25% of the fruit snack food product;
the pectin is present in an amount between approximately 1.5%-2.5% of the fruit snack food product; and
the one or more acids are present in an amount between approximately 0.1%-3% of the fruit snack food product.

13. The fruit snack food product of claim 12, wherein the fruit snack food product has a moisture content in the range of approximately 18%-30% and a Brix value in the range of 75° Bx to 83° Bx.

14. The fruit snack food product of claim 12, further comprising:
evaporated cane juice present in an amount between approximately 15%-25% of the fruit snack food product.

15. The fruit snack food product of claim 12, further comprising one or more salts present in an amount less than approximately 1%.

16. The fruit snack food product of claim 11, wherein:
the juice comprises white grape juice present in an amount between approximately 5%-15% of the fruit snack food product;
the tapioca syrup is present in an amount between approximately 25%-35% of the fruit snack food product;
the pectin is present in an amount between approximately 1.5%-2.5% of the fruit snack food product; and
the one or more acids are present in an amount between approximately 0.1%-3% of the fruit snack food product; and
the fruit snack food product further comprises tapioca solids in an amount between approximately 5%-15% of the food product.

17. The fruit snack food product of claim 16, wherein the fruit snack food product has a moisture content in the range of approximately 18%-30% and a Brix value in the range of 75° Bx to 83° Bx.

18. The fruit snack food product of claim 16, wherein the fruit snack food product further comprises evaporated cane juice in an amount between 25%-30% of the fruit snack food product.

19. The fruit snack food product of claim 11, wherein the PUFA comprises docosahexaenoic acid (DHA) present in an amount between 0.5%-1% of the fruit snack food product.

20. The fruit snack food product of claim 11, further comprising colorings and flavorings.
